(19) United States
(12) Patent Application Publication
(10) Pub. No.: US 2021/0190747 A1
(43) Pub. Date: Jun. 24, 2021

(54) MACHINE OLFACTION SYSTEM AND METHOD

(71) Applicant: X Development LLC, Mountain View, CA (US)

(72) Inventors: Bennet Joseph Cobley, Mountain View, CA (US); Rohinton Mehta, Fremont, CA (US); Radu Gogoana, Burlingame, CA (US)

(21) Appl. No.: 16/721,035

(22) Filed: Dec. 19, 2019

(51) Int. Cl. G01N 33/00

(52) U.S. Cl. CPC G01N 33/0031; G01N 33/0016; G01N 2021/177; G01N 33/0022; G01N 33/0073

(57) ABSTRACT

Methods, systems, and apparatus for a camera-enhanced multi-modal gas sensing apparatus including a camera configured to capture imaging data including at least a portion of a test environment including the gas sensing apparatus and an object of interest within the field of view of the camera, multiple gas sensors including a first type of gas sensor and a second type of gas sensor different from the first type of gas sensor which are each sensitive to a respective set of analytes, a housing configured to hold the multiple gas sensors, a gas inlet coupled to the housing and configured to expose the multiple gas sensors to a gas introduced from the test environment via the gas inlet, and a data processing apparatus in data communication with the multiple gas sensors and the camera.

### BACKGROUND

Gas sensor arrays can be used to detect the presence of analytes in ambient environments surrounding the gas sensors. Detecting particular analytes in an ambient environment, e.g., volatile organic compounds, can be useful for safety, manufacturing, and/or environmental monitoring applications. Individual gas sensors can be differently sensitized to a particular subset of analytes and nonreactive to other analytes.

### SUMMARY

This specification describes systems, methods, devices, and other techniques relating to a camera-enhanced multi-modal gas sensing array. The array of differently-sensitized gas sensors can be used to generate a recognizable pattern of output signals unique to a variety of analyte compositions to which the multi-modal gas sensor array is exposed. Visual input, e.g., from a camera, is utilized to enrich the gas sensing process for the multi-modal gas sensing apparatus.

In general, one innovative aspect of the subject matter described in this specification can be embodied in a multi-modal gas sensing apparatus including a camera configured to capture imaging data including at least a portion of a test environment including the gas sensing apparatus and an object of interest within the field of view of the camera. The apparatus includes multiple gas sensors including a first type of gas sensor and a second type of gas sensor different from the first type of gas sensor, where each of the first type of gas sensor and second type of gas sensor is sensitive to a respective set of analytes. The apparatus includes a housing configured to hold the plurality of gas sensors, a gas inlet coupled to the housing and configured to expose the multiple gas sensors to a gas introduced from the test environment via the gas inlet, and a data processing apparatus in data communication with the multiple gas sensors and the camera. The data processing apparatus is configured to perform the operations including receiving, from the camera, imaging data. The object of interest in the test environment and one or more object annotation labels are identified from the imaging data. Based on the object of interest and one or more object annotation labels, a proper subset of the plurality of gas sensors and a set of performance parameters is selected. The multiple gas sensors are exposed to a test gas from the test environment and, for each gas sensor of the proper subset of gas sensors, response data from the exposure to the test gas is collected.

These and other embodiments can each optionally include one or more of the following features. In some implementations, selecting the proper subset of the multiple gas sensors and the set of performance parameters includes selecting only the gas sensors that are sensitive to multiple analytes associated with the object of interest.

In some implementations the set of performance parameters includes an operating temperature of one or more of the proper subset of the multiple gas sensors. The set of performance parameters can include a sensitivity level of one or more of the proper subset of gas sensors.

In some implementations, selecting the set of performance parameters is based in part on one or more of a distance of the object of interest from the gas inlet, an air flow rate at the gas inlet, a relative toxicity of the object of interest, and a relative sensitivity of the multiple gas sensors to the object of interest. The distance of the object of interest from the gas inlet can be determined based on the imaging data including the object of interest.

In some implementations, the operations of the apparatus further include identifying, from the imaging data, one or more objects not of interest in the test environment and one or more object annotation labels for the objects not of interest, and selecting, based on the one or more objects not of interest and one or more object annotation labels for the objects not of interest, a modified proper subset of the multiple gas sensors and a modified set of performance parameters.

In some implementations, the operations of the apparatus further include identifying, based on the response data, one or more properties of the object of interest.

In some implementations, the apparatus includes a user interface including a touch-screen interface for a user to interact with the multi-modal gas sensing apparatus. User interaction can include identifying, by the user and by an indication on the touch-screen interface, one or more objects of interest in the field of view of the camera.

In general, another innovative aspect of the subject matter described in this specification can be embodied in methods for training a multi-modal gas sensor array including generating training data for multiple test gases, each test gas including multiple analytes and introduced into a first environment by an object of interest located within the first environment. For each test gas, the generating of training data including collecting, by a camera configured to capture the object of interest within a field of view of the camera, imaging data including the object of interest located within the first environment. The multi-modal gas sensor array including multiple gas sensors is exposed to the test gas, where the multiple gas sensors include a first type of gas sensor and a second type of gas sensor different from the first type of gas sensor. A set of sample data including response data for each of the multiple gas sensors is collected by a data processing apparatus and from each of the gas sensors responsive to the exposure of the test gas. A subset of gas sensors from the multiple gas sensors is selected from the set of sample data for the test gas, where the response data collected for each gas sensor of the subset of gas sensors meets a threshold response. Using the set of sample data, the imaging data is annotated by the data processing apparatus with an object annotation label. Training data for the test gas representative of the object of interest within the first environment is generated from the set of sample data and the labeled imaging data and provided to a machine-learned model.

These and other embodiments can each optionally include one or more of the following features. In some implementations, the object annotation label includes one or more of a distance of the object of interest from a gas inlet of the multi-modal gas sensor array, an air flow rate at the gas inlet, a relative toxicity of the object of interest, and a relative sensitivity of the plurality of gas sensors to the object of interest.

In some implementations, the methods further include collecting, by the camera, imaging data including a particular object of interest within the field of view of the camera located within a test environment, determining, by the data processing apparatus and from the imaging data, one or more object annotation labels for the particular object of interest, identifying, by the data processing apparatus and using the machine-learned model, a subset of gas sensors from the multiple gas sensors sensitive to one or more analytes associated with the particular object of interest based on the one or more object annotation labels, exposing the multi-modal gas sensor array including the multiple gas sensors to a test gas from the test environment including the particular object of interest, collecting, by the data processing apparatus and from the subset of gas sensors, response data from each of the subset of gas sensors identified as sensitive to the one or more analytes associated with the particular object of interest, and determining, by the data processing apparatus and using the machine-learned model, one or more characteristics descriptive of the particular object of interest within the test environment.

In some implementations, the one or more characteristics descriptive of the particular object of interest includes identifying respective concentrations of the one or more analytes associated with the particular object of interest.

In some implementations, determining the one or more object annotation labels for the particular object of interest includes determining a distance of the particular object of interest from the gas inlet of the multi-modal gas sensor array.

In some implementations, determining one or more object annotation labels for the object of interest includes performing image recognition analysis on the imaging data collected by the camera.

In some implementations, the methods further include receiving, from a user, a user interaction via a touch-screen interface of the multi-modal gas sensor array, wherein the user interaction includes identifying, by the user and by an indication on the touch-screen interface, one or more particular objects of interest in the field of view of the camera.

In some implementations, the methods further include determining, by the data processing apparatus and from the imaging data, one or more objects not of interest within the field of view of the camera, determining, by the data processing apparatus and from the imaging data, one or more object annotation labels for the one or more objects of not of interest, and identifying, by the data processing apparatus and using the machine-learned model, a modified subset of gas sensors from the plurality of gas sensors. The modified subset of gas sensors can be sensitive to one or more analytes associated with the particular object of interest based on the one or more object annotation labels for the object of interest, and can be not sensitive to one or more analytes associated with the one or more objects not of interest based on the one or more object annotations labels for the one or more objects not of interest.

In some implementations, identifying the subset of gas sensors from the multiple gas sensors sensitive to one or more analytes associated with the particular object of interest based on the one or more object annotation labels further includes selecting, by the data processing apparatus, performance parameters for the subset of gas sensors comprising an operating temperature of one or more of the gas sensors of the subset gas sensors. The set of performance parameters can include a sensitivity level of one or more of the gas sensors of the subset of gas sensors.

Particular embodiments of the subject matter described in this specification can be implemented so as to realize one or more of the following advantages. An advantage of this technology is that an optimized subset of the array of gas sensors in the multi-modal gas sensing apparatus can be selected to be sampled prior to the sensing process, e.g., the “sniff,” based in part on the visual information provided by the camera. This can reduce the data collected for a sniff and improve the performance of the apparatus in a test environment. Additionally, by adjusting one or more performance parameters based on the visual input, e.g., sensor sensitivity, baseline, time of sampling, sampling temperatures, gas flow rates, the collected data can have, for example, reduced signal-to-noise and optimized collection time. Utilizing visually-enhanced training data can assist in developing a machine-learned model that associates sight and smell, as well as building a high-contextual and effective software platform for operating the multi-modal gas sensing apparatus.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example system for training an e-nose gas sensing apparatus.

FIG. 2 is a block diagram of an example e-nose gas sensing apparatus.

FIG. 3 is a schematic of an example view of e-nose gas sensing apparatus.

FIG. 4 is a schematic of an example touch screen display of an e-nose gas sensing apparatus.

FIG. 5 is a flow diagram of an example process of the e-nose gas sensing apparatus.

FIG. 6 is a flow diagram of another example process of the e-nose gas sensing apparatus.

FIG. 7 is a block diagram of an example computer system.

Like reference numbers and designations in the various drawings indicate like elements.

### DETAILED DESCRIPTION

### Overview

The technology of this patent application utilizes visual input, e.g., from a camera, to enrich the gas sensing process for a multi-modal gas sensing apparatus.

More particularly, the technology customizes the operation of the gas sensing apparatus via environmental awareness utilizing imaging data collected by a camera configured to capture a portion of the sensing environment surrounding the gas sensing apparatus. In one aspect, the objects identified in the sensing environment from imaging data can be utilized to enhance the training data generated by the gas sensing apparatus for training a machine-learned model. Sensor response data for the multiple gas sensors in the multi-modal gas sensor array can be labeled with the objects identified visually in the sensing environment to generate training data with additional insight. An identified object and the sensor response data collected while the identified object is present in the sensing environment can be used to identify a proper subset of sensors of the multi-modal array of gas sensors to utilize for the particular object.

Real-time visual input from a camera or other imaging device prior to the sensing process can customize the operation of the gas sensing apparatus through software to optimize its performance. In particular, prior to the gas sensing process, image recognition software can be utilized to identify objects in the environment surrounding the gas sensing apparatus and to determine other information that can affect the performance of the gas sensing apparatus, e.g., relative distance of the object from the sensing apparatus, expected analytes generated by the object, potential confuser analytes not of interest in the environment, or other features of the objects and sensing environment. In one example, an appearance of the object of interest, e.g., a ripe banana versus a green banana, can affect the targeted analytes for the gas sensing apparatus. One or more performance parameters of the gas sensing apparatus can be adjusted to select a proper subset of the gas sensor array based in part on the visual input. Performance parameters define the operating conditions for each of the gas sensors, where each gas sensors may have a different set of adjusted variables that can be selected in response to the visual input. Performance parameters can include adjusting relative sensitivities of the array of gas sensors, setting a baseline, operating temperatures of the gas sensors, adjusting exposure thresholds, setting particular sampling temperatures, gas flow rates, sampling times, and the like.

Additionally, user provided context, e.g., via an interactive application or touch screen, can be utilized in combination with the visual input to enhance the sensing process. For example, a user may interact with an image displayed on an interactive touch screen for the gas sensing apparatus to indicate a particular object or region of the sensing environment that is of interest.

### E-Nose Multi-modal Gas Sensing Array Training Environment

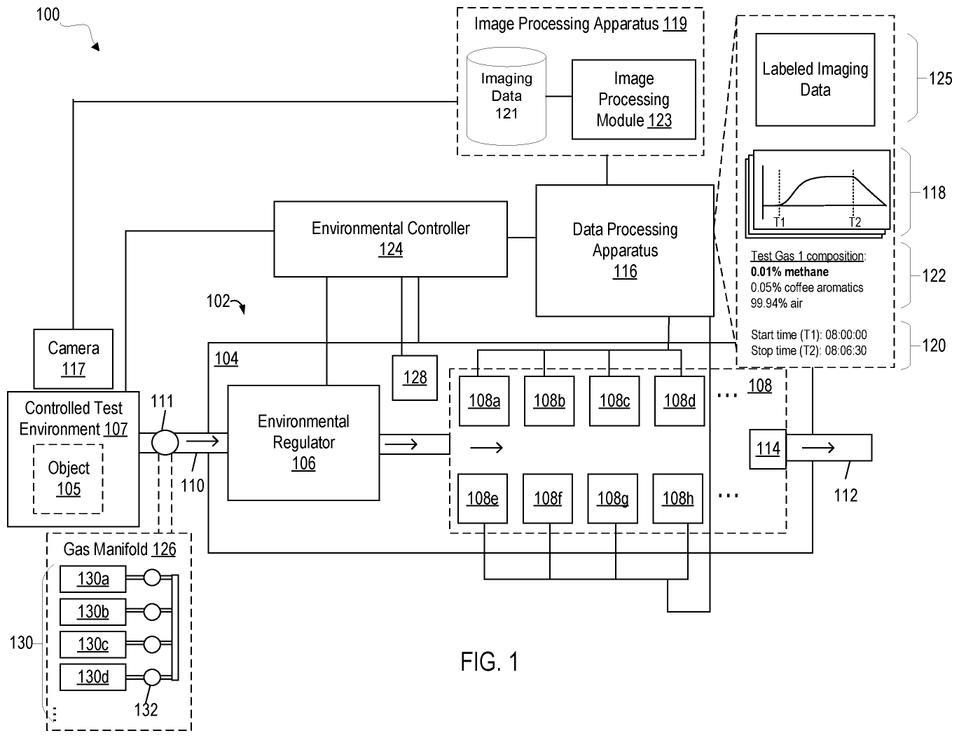

FIG. 1 is a block diagram of an example system 100 for training an e-nose gas sensing apparatus 102. The system 100 for training an e-nose gas sensing apparatus 102 can include a controlled environment, e.g., a laboratory setting, where external environmental factors, e.g., temperature, humidity, presence of chemicals/gases, is highly controlled and/or regulated.

The gas sensing apparatus 102 includes a housing 104 including an environmental regulator 106. Environmental regulator 106 can include a heat-exchange component, e.g., cylindrical heaters inserted into the housing, and/or heat transfer fins for controlling the temperature of gases that are introduced through the gas inlet 110. The heat-exchange component can be configured to interact with the gas and regulate a temperature of the gas to the particular temperature and prior to entering the gas inlet.

Environmental regulator 106 can be configured to control a temperature within the housing 104, gas sensors 108, and a gas within the housing 104, for example, to a temperature between 40-45° C., to a temperature above dew point >16° C., or at a temperature relevant to an environment of interest (e.g., room temperature 23° C.). In some implementations, environmental regulator 106 can be configured to regulate a relative humidity within the housing 104, gas sensors 108, and a gas within the housing 104, e.g., to a relative humidity below 10%, to a relative humidity between 10-30%, or to a relative humidity relevant to an environment of interest.

The housing 104 and environmental regulator 106 can be in thermal contact such that the gas introduced through the gas inlet 110, the gas sensors 108, and the housing 104 are all maintained at a same temperature during operation of the apparatus 102.

Housing 104 can be composed of various materials that are selected to be non-reactive to a set of analytes to which the housing 104 will be exposed. Materials for the housing 104 can include, for example, Teflon, Teflon-coated aluminum or stainless steel, Delrin, or other materials that are resistant to the set of analytes.

Housing 104 includes fixtures to hold a set of gas sensors 108 within the housing 104. The fixtures can be configured to accommodate particular dimensions of the gas sensors, and a layout of the fixtures within the housing 104 can be configured to designate particular locations for different types of gas sensors 108 within the housing 104.

Housing 104 further includes a gas inlet 110 and a gas outlet 112, where the gas inlet 110 is configured to allow for the introduction of gases into the housing 104 and to flow gas across the gas sensors 108. Gas outlet 112 is configured to allow for the purge of the gases from the housing 104.

Gas inlet 110 and gas outlet 112 can be configured for gas flow rates ranging between 0-10 cubic feet/hour, e.g., 5 cubic feet/hour. A particular flow rate for a gas into the gas inlet 110 can be selected, for example, based on an amount of time it takes for the environmental regulator 106 to bring a gas introduced at the gas inlet 110 to a test temperature, e.g., how long the gas will have to be in the fins to get it to the temperature of the housing 104.

In some implementations, gas sensing apparatus 102 includes a fan 114 configured to generate a negative pressure at the gas inlet 110 and within the housing 104 which can draw a gas into the housing 104 via the gas inlet 110, move the gas across the gas sensors 108, and purge the gas from the gas outlet 112. One or more operating parameters of the fan 114, e.g., a rotational speed of the fan, can be selected to regulate a desired flow rate of the gas through the gas sensing apparatus 102.

Gas sensors 108 include a multi-modal array of gas sensors that can be sensitive to various different organic and/or inorganic compounds. In other words, the multi-modal array of gas sensors 108 can include gas sensors that are responsive to certain analytes in a test gas and not sensitive to others. Types of gas sensors 108 can include gas sensors having different sensing mechanisms, e.g., metal oxide (MOx) sensors, photoionization detector (PID) sensors, electrochemical sensors, nondispersive infrared (NDIR) sensors, or other types of gas sensors. For example, gas sensors included in a gas sensing apparatus 102 include MOx sensors 108a-c, PID sensors 108d-f, electrochemical sensor 108g, and NDIR sensor 108h.

In some implementations, types of gas sensors 108 include gas sensors having a same sensing mechanism, e.g., oxidation-based, resistivity-based, optical-based, etc., but can have different sensitivities to the multiple analytes. In other words, a first type of gas sensor 108a and a second type of gas sensor 108b have a same mechanism for gas sensing, e.g., MOx sensors that make resistivity-based measurements, but are configured to have different performance parameters, e.g., a MOx sensor operating at a first voltage bias and a MOx sensor operating at a second voltage bias, such that the respective sensitivities to certain analytes are different.

The multi-modal array of gas sensors 108 can include multiple gas sensors, e.g., 38 total gas sensors, 25 total gas sensors, greater than 40 total gas sensors. A number of each type of gas sensor relative to respective numbers of each other type of gas sensor included in the multi-modal array of gas sensors 108 can depend in part on dimensional/size considerations, cost-benefit of each type of sensor, responsivity, signal-to-noise ratio of each sensor, or the like. A field of application of the sensor array, e.g., agricultural, industrial, etc., can determine a number of each type of gas sensor relative to a respective number of each other type of gas sensor in the multi-modal array of gas sensors 108. For example, for applications that may have a fainter signal-to-noise ratio, e.g., more background analytes not of interest, more sensors will be included overall in the multi-modal array of gas sensors 108.

In some implementations, multiple different gas sensors can each have a same sensitivity to a particular analyte, e.g., a first type of gas sensor and a second type of gas sensor can each be sensitive to the particular analyte.

The array of gas sensors 108 can be configured within the house 104 such that a test gas introduced via an inlet 110 can be sampled simultaneously by all of the gas sensors 108 in the array of gas sensors. In other words, the test gas is exposed to the multiple gas sensors in the array of gas sensors simultaneously such that data collection from each of the multiple gas sensors 108 can be performed in parallel.

In some implementations, the multi-modal array of gas sensors can include multiple MOx sensors 108a-c that are each configured to operate at a different temperature, e.g., biased at a different operating voltage, such that they have different responses to particular analytes based on the operating parameters.

Each gas sensor 108 is connected to a data processing apparatus 116 which is configured to collect data from the gas sensors 108, e.g., response data 118. Response data 118 can include a time-dependent response of each of the gas sensors 108 to a test gas. Response data 118 can include a measure of the resistivity of the gas sensor 108 versus time.

Additionally, the data processing apparatus 116 can be configured to collect time-dependent measurements of operating conditions of the plurality of sensors 108, gas inlet 110, and environmental controller 124, e.g., temperature and relative humidity, gas flow rate, and the like.

System 100 additionally includes an imaging device, e.g., a camera 117, and an image processing apparatus 119. In some implementations, the operations performed by the image processing apparatus 119 can be performed additionally or entirely by the data processing apparatus 116. Camera 117 is an imaging device configured to capture image/video data of an object 105 within a controlled test environment 107 within a field of view of the camera 117. Camera 117 can be, for example, a CCD camera, CMOS camera, or the like. In some implementations, camera 117 can collect imaging data and audio data. Camera 117 can include one or more additional filters, e.g., an infrared filter, to measure a temperature of the object 105 and/or controlled test environment 107.

Imaging data 121 captured by the camera 117 of the object 105 in the controlled test environment 107 can be processed by the image processing apparatus 119 including image processing module 123. Processing of the imaging data 121 can include identifying, within the imaging data 121, one or more objects 105 within the controlled test environment 107. An object 105 identified within the imaging data 121 can be an object of interest, e.g., a user of the system 100 may be interested in performing a “sniff” of the object and its associated analytes, or an object not interest, e.g., a confounding object. In one example, imaging data 121 may capture the controlled test environment 107 including several fruits, e.g., a banana and an apple, as well as several other objects not of interest, e.g., a container of coffee. Image processing can be performed on the imaging data 121 by the image processing module 123 to identify each of the objects 105 in the controlled test environment 107 and captured by the imaging data 121. Additionally, one or more object annotation labels are identified for each object 105 in the imaging data 121.

In some implementations, object annotation labels can be descriptive terms related to the object 105 including physical characteristics of the object. For example, in the case of a banana-type object, the object annotation labels can be “ripe,” “unripe,” “green,” “yellow,” or the like. Object annotation labels can include relative distances of the object 105 from the gas inlet 110, e.g., a relative location within the controlled test environment 107 and/or one or more dimensions of the object 105.

In some implementations, identifying objects characteristics can include applying a set of classifiers to the object 105. Classifiers can be utilized to sort an object into a general category and identify a set of common analytes associated with the category. For example, an object 105 can be classified as a fruit and a set of common analytes for fruits can be identified.

The object annotation labels can be applied by the image processing module 123 to the imaging data 121 to generate labeled imaging data 125. The labeled imaging data 125 can then be associated with the response data collected by the gas sensors 108 of apparatus 102 to generate training data for the multi-modal gas sensor apparatus 102, as discussed in more detail below.

Data processing apparatus 116 can be hosted on a server or multiple servers in data communication with the gas sensors over a network. The network may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. The network may include multiple networks or subnetworks, each of which may include, for example, a wired or wireless data pathway. The network may include a circuit-switched network, a packet-switched data network, or any other network able to carry electronic communications (e.g., data or voice communications). For example, the network may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. The network may include one or more networks that include wireless data channels and wireless voice channels. The network may be a wireless network, a broadband network, or a combination of networks including a wireless network and a broadband network.

Data processing apparatus 116 can be configured annotate the response data 118 received from the gas sensors 108 responsive to a test gas. In some implementations, annotation data 120 includes timestamps, e.g., a start time label, a stop time label, respective composition data 122 of the test gases, e.g., a set of known analytes of interest and set of known analytes not of interest, being evaluated using the gas sensing apparatus 102, and the labeled imaging data 125 generated by the image processing apparatus 119.

In some implementations, the data processing apparatus 116 is configured to generate annotation data 120 before, during, and after exposure of a test gas to the gas sensors 108, where the annotation data 120 includes recording a first label describing a first state of the multi-modal gas sensing array, e.g., a start time of the gas exposure.

Exposure of the gas sensors 108 to the test gas can include placing an object 105 within a controlled test environment 107. A test object 105 having one or more associated analytes, e.g., having one or more measureable aerosolized chemical compounds, can be placed within the controlled test environment 107, e.g., a temperature/humidity controlled environment with minimized additional analyte exposure, such that the controlled test environment 107 is in fluid communication with the gas inlet 110. A valve 111 located between the controlled test environment 107 and the gas inlet 110 can be used to regulate exposure of the object 105 to the gas sensors 108, e.g., start a gas exposure and terminate a gas exposure.

The data processing apparatus 116 can then collect a set of sample data 118 from each of the gas sensors 108 of the multi-modal gas sensing array responsive to the test gas from the object 105 in the controlled test environment 107 and then record annotation data 120, e.g., a second label, describing a second state of the multi-modal gas sensing array, e.g., a stop time of gas exposure.

In some implementations, instead of or in addition a test gas source from the object 105 located in the controlled test environment 107, a gas manifold 126 can provide a first test gas to the multi-modal array of gas sensors 108, where the first test gas includes a first concentration of a known analyte of interest and a second concentration of a known analyte that is not of interest, e.g., a confounding gas. Further details of the gas manifold 126 are found below.

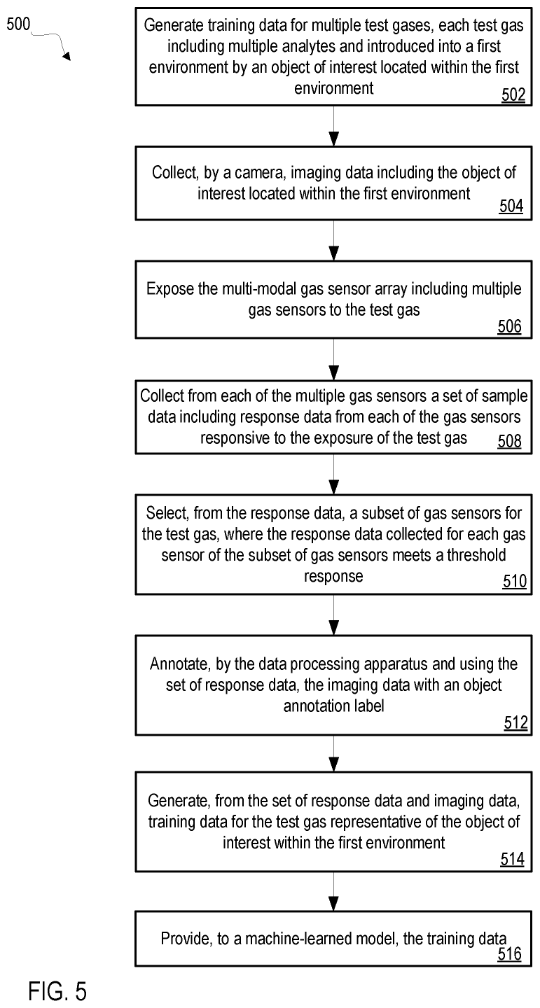

In some implementations, the data processing apparatus 116 further generates, from the response data 118, the annotation data 120, the composition data 122, and the labeled imaging data 125 for the known test gases associated with the objects 105, training data for a machine-learned model. Details described in further detail below with reference to FIG. 5.

In some implementations, the data processing apparatus 116 is in data communication with an environmental controller 124. The environmental controller 124 may be a part of the data processing apparatus 116. The environmental controller 124 can be configured to provide operating instructions to the environmental regulator 106, gas sensors 108, data processing apparatus 116, controlled test environment 107, and gas manifold 126. In some implementations, the environmental controller 124 can be configured to receive operational feedback, e.g., solenoid valve status, temperature control, etc., from one or more of the environmental regulator 106, gas sensors 108, data processing apparatus 116, controlled test environment 107, and gas manifold 126.

In some implementations, the environmental controller 124 can receive operating conditions feedback, e.g., temperature, humidity readings, from a temperature and/or humidity gauge 128. The gauge 128, e.g., a thermocouple, hygrometer, or the like, can be in physical contact with the housing 104 or gas sensors 108 to measure a temperature and/or relative humidity. The gauge 128 can additionally or alternatively measure a temperature and/or relative humidity of the gas present within the housing 104.

In some implementations, the operating conditions feedback received by the environmental controller 124 can be provided to the data processing apparatus 116 and recorded as annotation data 120. For example, temperature of the housing 104, gas sensors 108, the controlled test environment 107, and test gas can be recorded as annotation data 120 and included with the response data 118 for the particular test gas.

In some implementations, the system 100 further includes a gas manifold 126 having multiple gas sources 130. The multiple gas sources 130 can each include multiple known analytes of interest and multiple known analytes not of interest. For example, gas manifold 126 can include gas source 130a, 130b each having analytes of interest and gas sources 130c and 130d each having analytes not of interest (e.g., confounding gases).

Gas manifold 126 can be a closed-loop system, where no external compounds are present within the gas manifold 126 that could interfere with the test gases provided by the gas manifold to the gas sensing apparatus 102 via the gas inlet 110. The test gases provided by the gas manifold 126 to the gas sensing apparatus 102 can be composed only of the known analytes of interest and known analytes not of interest from the one or more gas sources 130.

Gas manifold 126 can further include regulatory components 132 which can be operable to selectively allow a controlled flows (e.g., 0-5 cubic feet/hour) of one or more of the gas sources 130 from the gas manifold 126 and into the gas inlet 110 to generate a particular test gas for the gas sensing apparatus 102.

For a particular test gas generated by an object 105 in the controlled test environment 107 and/or provided by the gas manifold through the gas inlet 110 of the e-nose gas sensing apparatus 102, response data 118 is collected from each of the gas sensors 108 of the gas sensing array. The gas sensors 108 can be a multi-modal array of gas sensors having a variety of response characteristics to a range of analytes. Different types of gas sensors 108 can be more or less responsive to a particular analyte, and a subset of the gas sensors 108 can be identified as optimally responsive to the particular analyte based in part on the response data 118 collected.

In some implementations, response of a gas sensor 108 to a test gas including one or more analytes can be measured as a change in gas sensor electrical resistivity before, during, and after exposure to the particular test gas (e.g., as in MOx sensors). In some implementations, response of a gas sensor 108 can be measured as an electrical signal generated by one or more analytes of interest that are present in the test gas (e.g., as in PID sensors).

### E-Nose Multi-Modal Gas Sensing Apparatus

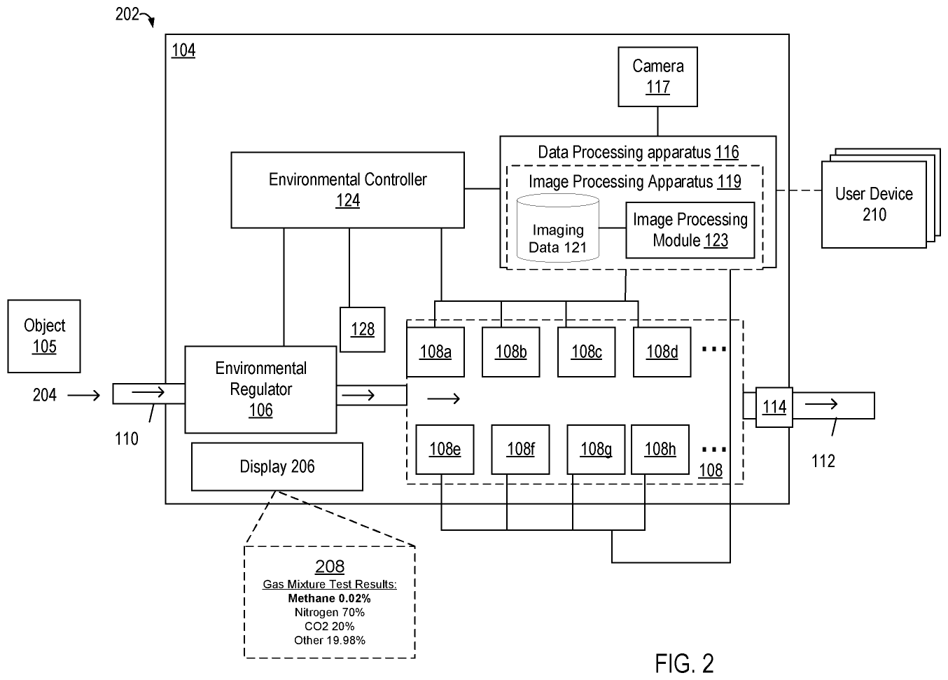

FIG. 2 is a block diagram of an e-nose gas sensing apparatus 202. As discussed above with reference to FIG. 1, the e-nose gas sensing apparatus 202 includes a housing 104 including a gas inlet 110 configured to receive an unknown gas mixture 204 and a gas outlet 112 through which the unknown gas mixture 204 is purged from the e-nose gas sensing apparatus 202.

The gas inlet 110 is coupled to the housing 104 and configured to expose the multiple gas sensors 108 to an unknown gas mixture 204 that is introduced through the gas inlet 110. Unknown gas mixture 204 can be introduced passively via the environment surrounding the gas inlet 110, e.g., from an object 105 in the environment of the gas sensing apparatus 202. For example, the unknown gas mixture 204 can be introduced via the gas inlet 110 when the gas sensing apparatus 202 is deployed in a testing environment, e.g., in a factory setting. Passive introduction of the unknown gas mixture 204 into the gas inlet 110 can be, for example, by diffusion of the unknown gas mixture into the gas inlet 110.

In some implementations, the unknown gas mixture 204 can be introduced actively into the gas inlet 110, for example, by generating a negative pressure within the housing 104 using a fan 114 or other similar device. In another example, the unknown gas mixture 204 can be introduced actively to the gas inlet 110 by a positive pressure of the gas at the gas inlet, e.g., by a person blowing into the gas inlet 110, a gas exhaust from a piece of equipment, an object 105 in proximity to the gas inlet 110, or the like.

Multiple gas sensors 108 including a first type of gas sensor, e.g., gas sensor 108a, and a second type of gas sensor, e.g., gas sensor 108b, that is different from the first type of gas sensor are located within the housing 104 where each of the first type of gas sensor and second type of gas sensor is sensitive to a respective set of analytes. The first type of gas sensor 108a and the second type of gas sensor 108b can have different methods for gas sensing, e.g., where the first type of sensor is a MOx sensor and the second type of sensor is a PID sensor.

In some implementations, the first type of gas sensor 108a and the second type of gas sensor 108b have a same method for gas sensing but are configured to have different performance parameters, e.g., a MOx sensor operating at a first operating temperature and a MOx sensor operating at a second operating temperature. Operating the MOx sensors at different temperatures can cause each respective MOx sensor to respond differently to analytes in a test gas, even when sensing a same test gas at a same concentration.

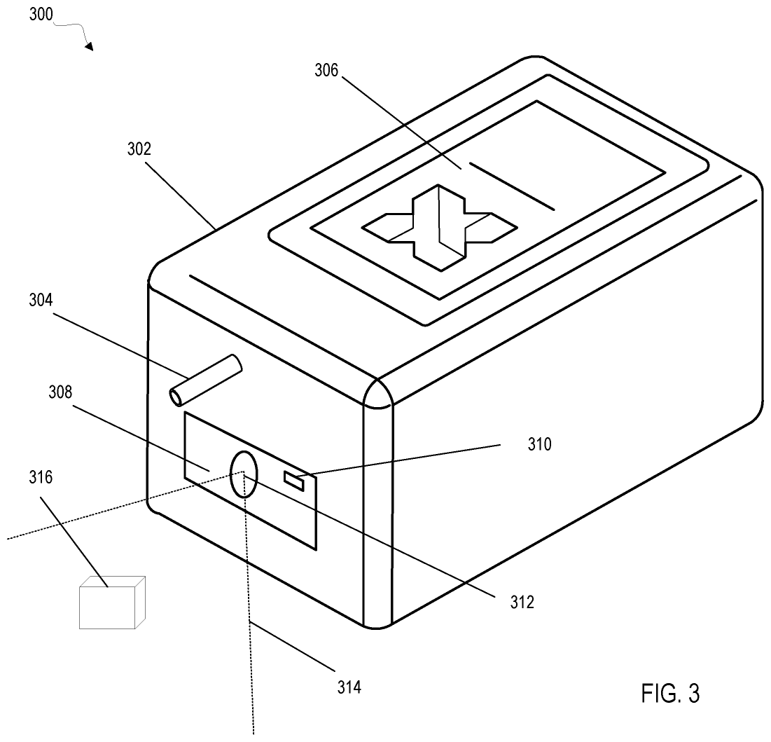

Gas sensing apparatus 202 further includes a camera 117 configured to capture imaging data including at least a portion of a test environment including the gas sensing apparatus 104 and an object of interest 105 within the field of view of the camera 117. As described with reference to FIG. 1, data processing apparatus 116 can receive, from the camera 117, imaging data including at least a portion of the test environment and an object of interest 105 and determine, from the imaging data, one or more object annotation labels of the object of interest 105. An example orientation of the camera 117 with respect to the housing 104 of the gas sensing apparatus 202 is described below with reference to FIG. 3.

Gas sensing apparatus 202 further includes an environmental controller 124 coupled to the housing 104 and configured to regulate temperatures of the housing 104, gas inlet 110, and gas sensors 108 to a particular temperature. The environmental controller 124 can include an environmental regulator 106, for example, a heating source and heat-transfer fins, embedded into the housing 104 where gas introduced at the gas inlet 110 passes through heating channels within the heat-transfer fins of the housing 104 to stabilize a temperature of the gas 204 to the particular temperature. In some implementations, the environmental controller 124 can include temperature and/or humidity gauges 128 to measure a temperature and/or relative humidity of the unknown gas mixture 204 that is received at the gas inlet 110.

Gas sensing apparatus 202 includes a data processing apparatus 116 in data communication with the gas sensors 108 and the environmental controller 124. The data processing apparatus 116 can be an onboard computer that is affixed to the housing 104 of the apparatus 202. In some implementations, a portion or all of the data processing apparatus 116 can be hosted on a cloud-based server that is in data communication with the gas sensing apparatus 202 over a network.

Data processing apparatus 116 can include a user device 210, where a user can interact with the gas sensing apparatus 202 via the user device 210, e.g., receive data, provide testing instructions, receive testing information, or the like. User device 210 can include, for example, a mobile phone, tablet, computer, or another device including an application environment through which a user can interact with the gas sensing apparatus 202. In one example, user device 210 is a mobile phone including an application environment configured to display gas mixture test results for the unknown gas mixture 204, allow for user interaction with the gas sensing apparatus 202, and the like.

In some implementations, the apparatus 202 includes a display 206 configured to communicate information 208 to a user of the gas sensing apparatus 202 and/or allow for user interaction with the gas sensing apparatus 202. Information 208 can include, for example, the operational status of the apparatus 202, e.g., on/off, testing, processing, etc.

In some implementations, information 208 includes test results for the unknown gas mixture 204. Information 208 can be presented to a user based on user preferences, e.g., to highlight a particular set of analytes that the user is interested in discovering in the unknown gas mixture 204. Information 208 can be additionally or alternatively provided to one or more user devices 210 in data communication with the apparatus 202.

In some implementations, display 206 is configured to respond to user interaction, e.g., a touch-screen functionality. Display 206 can further include audio feedback, e.g., an alert, to notify a user of the status of the apparatus 202. For example, the apparatus 202 can provide an audio and/or visual update to the user of a testing status. In another example, the apparatus 202 can provide an audio and/or visual alarm to the user, e.g., if a particular analyte is detected above/below a preset threshold, e.g., a threshold concentration of the analyte is detected in the ambient.

In some implementations, imaging data can be captured of an environment surrounding the apparatus 202, e.g., using a camera or video recording device. The imaging data of the surrounding environment can be displayed on display 210 to identify, to a user, one or more objects 105 in the surrounding environment that may be included in the unknown gas mixture 204 being sampled by the apparatus 202. A blend ratio of the various analytes that are being sensed in the unknown gas mixture 204 can be displayed on display 210.

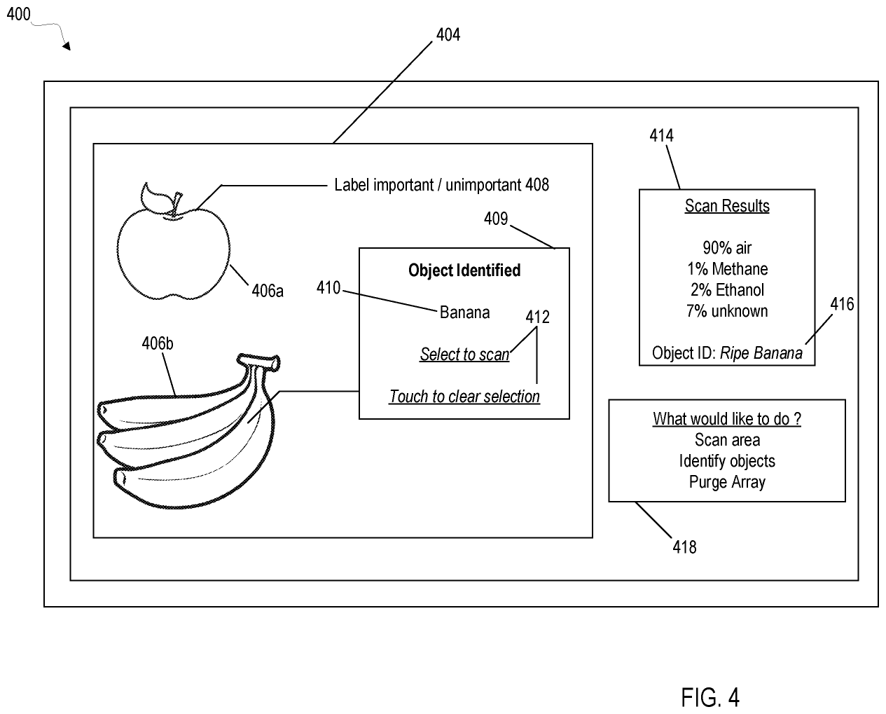

In some implementations, a user can interact with the displayed imaging data on display 206 to select a particular object 105 and identify the particular object 105 as an object of interest. The data processing apparatus 116 can receive the user input via a touch screen of the display 206 and, in response, adjust one or more sensing parameters, e.g., selecting the subset of gas sensors 108 to utilize for a “sniff” in response to a particular object of interest. Further details of the display 206 are disclosed below with reference to FIG. 4.

FIG. 3 is a schematic of an example view of e-nose gas sensing apparatus 300, e.g., gas sensing apparatus 202. The gas sensing apparatus 300 can include housing 302, e.g., housing 104, enclosing the various components of gas sensing apparatus 300. A gas inlet 304, e.g., gas inlet 110, can receive an unknown gas mixture, e.g., unknown gas mixture 204, and provide the unknown gas mixture to a multi-modal gas sensing array, e.g., multi-modal gas sensing array 108, within the housing 302. Gas sensing apparatus 300 can include a display 306, e.g., display 206, which can include a touchscreen. Display 306 can provide an intuitive user interface for receiving user instructions, e.g., run test, testing parameters, etc., and display information, e.g., information 208, to a user viewing the display 306.

Gas sensing apparatus 300 further includes an imaging device 308, e.g., camera 117. The imaging device 308 can include a light source 310, e.g., a flash bulb, light emitting diode, or the like, for providing illumination of a region surrounding the apparatus 300 including a field of view 314 of the camera 117. An aperture and/or lens 312 of the imaging device 308 can be located on a surface of the housing 302 such that the field of view 314 of the imaging device 308 captures a portion of the area surrounding the gas sensing apparatus 300.

In some implementations, the aperture and/or lens 312 of the imaging device 308 can be selected such that a field of view 314 of the imaging device 308 extends to a substantial region surrounding the housing 302, e.g., a wide-angle lens. A location of the aperture and/or lens 312 of the imaging device 308 with respect to the housing 302 can be selected to maximize a field of view 314 of the imaging device 308.

As depicted in FIG. 3, the field of view 314 of the imaging device 308 includes an area including a region surrounding the gas inlet 304. In particular, objects of interest 316, e.g., object 105, that are adjacent or nearby to the gas inlet 304 can be captured by the imaging device 308 within the field of view 314 of the imaging device 308. Multiple objects 316 can be captured in imaging data collected by the imaging device 308, as described in further detail with reference to FIGS. 1 and 2 above.

In some implementations, a location of the imaging device 308 with respect to the housing 302 can be adjustable, e.g., using a mechanical tip/tilt, translation, or other mount. The location of the imaging device 308 may be automatically adjustable, e.g., by data processing apparatus 116, to capture different fields of view 314 of the region surrounding the housing 302.

A footprint, e.g., width and length, of the gas sensing apparatus 300 can be, for example, smaller than 2×4 inches, smaller than 10×12 inches, smaller than 20×20 inches, or the like. Though depicted in FIG. 3 as having a rectangular form factor, other form factors are possible, e.g., cylindrical form factor. In one example, the gas sensing apparatus 300 can have the dimensions similar to a standard shoe box, e.g., 14×8×5 inches. In some implementations, a footprint of the gas sensing apparatus 300 can be fit to the dimensions of a silicon chip, e.g., on the order of 1 mm×1 mm×0.1 mm or smaller.

FIG. 4 is a schematic of an example touch screen display 400 of an e-nose gas sensing apparatus. Touch-screen display 400, e.g., display 306, can include a imaging data 404 including real-time imaging of an area surrounding the gas sensing apparatus, e.g., gas sensing apparatus 300.

In some implementations, objects 406a and 406b, e.g., objects 316, captured within a field of view of the camera, e.g., field of view 314 of camera 308, are identified by the data processing apparatus 116. The objects 406a and 406b may be labeled in the imaging data 404 presented on the display 400, where the labeling of the objects 406a and 406b can include requests for further input from a user, e.g., label request 408 for object 406a.

In some implementations, an object 406b may be identified by the data processing apparatus 116, where one or more object annotations 410 are presented with the object 406b, e.g., the label “banana,” as depicted in FIG. 4. Object annotations 410 can be generic categories and/or more specific identifiers based on the imaging data 404 including the object 406b.

Additional selectable options 412, e.g., “select to scan” and “touch to clear selection,” may be identified in window 409, where a user of the gas sensing apparatus can select one or more of the selectable options 412.

In some implementations, a user selection of “select to scan” option 412 can trigger the gas sensing apparatus to initiate a measurement by the gas sensing apparatus, e.g., a “sniff,” using a particular subset of gas sensors 108 that are selected based in part on the identified object 406b in the imaging data 404, as described in further detail below with reference to FIG. 5. For example, a subset of gas sensors 108 that are sensitive to organic analytes, in particular analytes that are known to be emitted by bananas, can be selected to collect response data in order to determine composition data for the object 406b.

In some implementations, display 400 can include scan results 414 that include, for example, composition data for the test gas sampled by the gas sensing apparatus of the environment surrounding the gas sensing apparatus and including the object 406b. Composition data can identify the one or more analytes detected in the test gas as well as a concentration of each analyte in the test gas.

In some implementations, one or more properties of the selected object 406b can be identified based on the response data and/or imaging data 404. For example, the selected object 406b can be identified in the scan results 414, based in part on the imaging data 404 and the response data generated by the gas sensors 108. An object ID 416 can be presented in the display 400 to the user which identifies one or more specific object annotations of the object 406b, e.g., “ripe” as an object annotation of object “banana.”

In some implementations, other user inputs are possible via, for example, a user input window 418 as depicted in FIG. 4. Other user inputs can include, for example, a selection to scan the area surrounding the gas sensing apparatus, a selection to identify objects within the field of view of the camera and using the imaging data 404, and a selection to purge the gas sensing array 108.

One or more of the functions described with respect to the display 400 can be performed on a secondary device, e.g., a user device. For example, a user may interact with the gas sensing apparatus using a mobile phone, tablet, computer, or the like. An application environment on the user device may be configured to display similar information and options as described with reference to display 400 in FIG. 4.

### Example Operation of E-Nose Multi-Modal Gas Sensing Apparatus

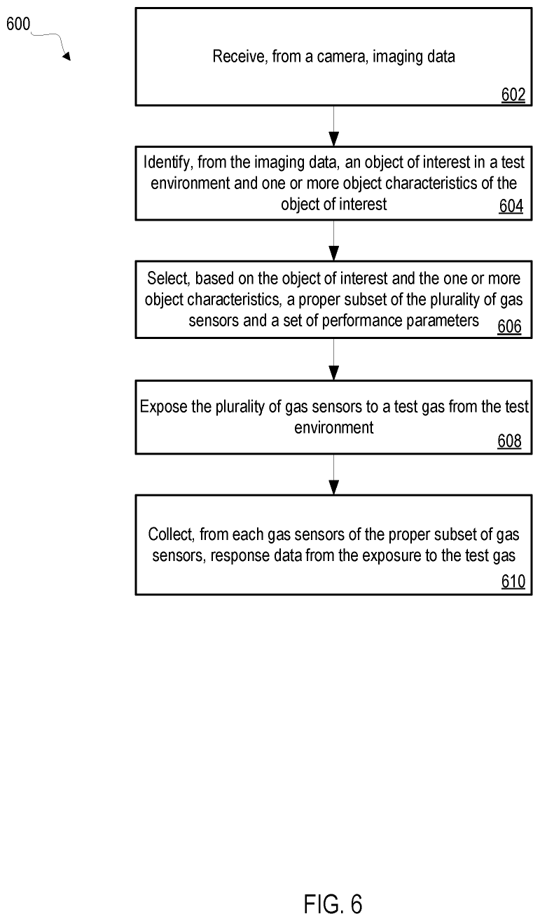

The e-nose multi-modal gas sensing apparatus can operate in various modes, including a training mode, e.g., training a machine-learned model to identify various analytes of interest, as described in detail with reference to FIG. 5, and detection mode, e.g., where the e-nose multi-modal gas sensing apparatus is deployed in a testing environment, as described in detail below with reference to FIG. 6.

FIG. 5 is a flow diagram of an example process 500 of the e-nose gas sensing apparatus. The e-nose gas sensing apparatus 102 can be trained using system 100 including multiples test gases each including multiple analytes from multiple objects 105 and imaging data 121 collected by camera 117 of the objects 105 within a controlled test environment 107.

Training data can be generated using system 100 and provided to train a machine-learned model which can then be deployed in a test environment to detect one or more analytes. Multiple sets of training data can be generated, where each set of training data can be customized for a particular test environment, e.g., a factory environment, an agricultural environment, a home environment, etc., such that the machine-learned model is trained to recognize a set of objects 105 with associated analytes that are relevant to the environment, e.g., objects commonly found in a fabrication environment, and of importance to the particular environment, e.g., detecting objects associated with toxic chemicals rather than inert chemicals. The process 500 described with reference to FIG. 5 is flexible such that the training data can be generated using a same gas sensing apparatus 102 for multiple different environments using a different set of objects with associated known analytes and known concentrations of the analytes from objects 105 located in a controlled test environment 107.

Training data is generated for multiple test gases, each test gas including multiple analytes and introduced into a first environment by an object of interest located within the first environment (502). The first environment, e.g., an industrial environment, an agricultural environment, a residential environment, etc., can have a particular set of relevant objects with associated analytes of interest and analytes not of interest, depending on the particulars of the environment. The objects 105 each include an associated composition of multiple analytes, where the composition can include known concentrations of a subset of analytes of interest and analytes not of interest that are emitted and detectable from the object 105.

For each test gas the generating of training data includes collecting, by a camera configured to capture the object of interest within a field of view of the camera, imaging data including the object of interest located within the first environment (504).

As described above with reference to FIG. 1, a camera 117 can be positioned with respect to the controlled test environment 107 to capture within a field of view of the camera 117 at least a portion of controlled test environment 107 including an object 105. In particular, the field of view of the camera 117 can include an area including the gas inlet 110, where an object 105 located within a controlled test environment 107 can be detected in the field of view of the camera 117. Imaging data 121 can be collected from the camera 117 including the object 105 within the field of view of the camera, where the imaging data 121 can be processed, e.g., using imaging processing apparatus 119 to identify the object 105 in the imaging data 121.

Imaging data 121 can include multiple objects 105 within the field of view of the camera, including objects of interest and objects not of interest. Each of the objects 105 captured by the imaging data can be a source of a respective test gas including multiple analytes. For example, an object 105 is a banana where a test gas including multiple volatile organic compounds (VOCs), e.g., ethylene, is emitted by the banana and measureable by the gas sensing apparatus 102.

Imaging data 121 captured by camera 117 can be provided to the image processing module 123 to identify and label the objects 105 captured in the imaging data 121. Image processing software can be utilized to process the imaging data 121 and identify the objects 105.

The imaging data 121 can further be analyzed using image processing module 123 to identify one or more object annotation labels. Object annotation labels can include physical characteristics, e.g., size, dimensions, colors, or other physical attributes, for the objects 105 captured in the imagine data 121. Object annotation labels can include one or more categories for the object, e.g., “fruit” and “ripe” for a banana object. In some implementations, object annotation labels can include information descriptive of a relative location of the objects with respect to the field of view of the camera and/or relative location of the objects with respect to a gas inlet 110 of the gas sensing apparatus 202. For example, an object annotation label may identify that the object is 3 feet away from the gas inlet 110 or that the object is 2 inches from the gas inlet 110.

The multi-modal gas sensor array including multiple gas sensors is exposed to the test gas, where the multiple gas sensors include a first type of gas sensor and a second type of gas sensor different from the first type of gas sensor (506). Exposing the multiple gas sensors 108 to the test gas can include exposing the gas sensors 108 to the object 105 in the controlled test environment 107, e.g., by opening a valve 111 to allow the test gas from the object 105 to flow into gas inlet 110. A negative pressure across the gas sensors 108 generated by fan 114 can be utilized to pull the test gas emitted from the object 105 across the gas sensors 108 in the apparatus 104.

In some implementations, providing the test gas of known analytes to the multiple gas sensors 108 includes providing the test gas in a controlled environment including a particular temperature and a particular relative humidity. An environmental regulator, e.g., environmental regulator 106 including heat transfer fins, can be used to alter a temperature and/or relative humidity of the test gas prior to the test gas reaching the multiple gas sensors 108. Temperature and humidity of the test gas can be regulated, for example, to room temperature and a relative humidity below dew point.

In some implementations, providing the test gas to the gas sensors 108 includes providing the test gas at a flow rate of less than 5 cubic feet per hour for a known period of time. A flow rate can be controlled, for example, using a negative pressure from fan 114 to draw the test gas from the controlled test environment 107 across the gas sensors 108. A flow rate can be, for example, as low as 1 cubic centimeter per minute. In another example, a flow rate can be up to 2 liters per minute. A particular flow rate can be selected based in part on a desired temperature and/or relative humidity of sampling and a temperature and/or relative humidity of the test gas prior to entering the gas inlet 110. In other words, an amount of time required to regulate the temperature and/or relative humidity of the test gas prior to exposure to the gas sensors 108 by the environmental regulator 106, e.g., heating fins, can determine a flow rate of the test gas within the environmental regulator 106.

In some implementations, exposing the multi-modal gas sensor array includes flowing a test gas from a gas manifold 126 through gas inlet 110 into the environmental regulator 106. Within the environmental regulator 106, e.g., heating/cooling fins, the test gas is regulated to a particular temperature, e.g., as measured by temperature gauge 128 and monitored by environmental controller 124. The test gas is then provided to the array of gas sensors 108 and exhausted through gas outlet 112. A flow of the test gas within house 104 can be controlled by the environmental controller 124 and regulated in part using regulatory components of the gas manifold 126, e.g., a flow meter and/or pressure regulator, by a negative pressure generated by fan 114 within the housing 104, or a combination thereof.

A set of sample data including response data for each of the multiple gas sensors responsive to the exposure of the test gas is collected by a data processing apparatus and from each of the multiple gas sensors (508). As depicted in FIG. 1, multiple gas sensors 108 including a first type of gas sensor 108a and a second type of gas sensor 108b can be a different type of sensor. For example, gas sensor 108a can be a MOx sensor and gas sensor 108b can be an electrochemical gas sensor. In another example, gas sensor 108a can be a MOx sensor operated at a first voltage bias and gas sensor 108b can be a MOx sensor operated at a second, different voltage bias.

In some implementations, the first type of gas sensor can be an organic-type gas sensor and the second type of gas sensor can be an inorganic-type gas sensor. For example, a volatile organic compound (VOC) sensor is sensitive to organic compounds, e.g., hydrogen, carbon dioxide, etc. In another example, PID sensors are sensitive to inorganic compounds, e.g., chlorine, tin oxide, etc.

Response data 118 can be collected by data processing apparatus 116 from each of the multiple gas sensors 108 of the gas sensing apparatus 102. The response data 118 can include multiple different formats of responses depending in part on a type of gas sensor 108 of the multiple different types of gas sensors. Formats of response data can include optical response data, e.g., from PID sensors, electrical resistivity data, e.g., from MOx sensors, and oxidation/reduction response data, e.g., from electrochemical sensors. In some implementations, the response data 118 includes a measure of electrical resistivity of the gas sensor over a period of time during the exposure to the test gas, e.g., for MOx gas sensors.

In some implementations, response data includes a response of the gas sensor 108 over a period of time. In one example, response data includes a measure of a change in electrical resistivity of the gas sensor over time.

Each of a first type of gas sensor and the second type of gas sensor can have a different response to the multiple known analytes of the test gas. For example, an organic-type gas sensor may react to an organic analyte, e.g., methane, in the test gas and not react to an inorganic analyte, e.g., tin oxide, in the test gas, and an inorganic-type gas sensor may not react to the organic analyte in the test gas and react to the inorganic analyte in the test gas.

Additionally, annotation data 120, e.g., timestamps, temperature/humidity data, etc., delineating when the test gas is exposed to the gas sensors 108 can be recorded, e.g., when the test gas is provided from the environmental regulator 106 to the array of gas sensors 108.

The sample data can include labeled imaging data 125, where the labeled imaging data includes objects 105 identified within the imaging data 121 collected by camera 117 of the controlled test environment 107.

Composition data 122 describing a particular composition of the test gas including the respective concentrations of one or more analytes of interest and one or more analytes not of interest in the test gas can be recorded for each test gas. The known analytes of interest and known analytes not of interest for the test associated with the object 105 in the controlled test environment 107 can be associated with the response data 118 generated by each gas sensor of the multi-modal array of gas sensors 108.

A subset of gas sensors are selected using the sample data for the test gas, where the response data collected for each gas sensor of the subset of gas sensors meets a threshold response (510). The subset of gas sensors can be selected, for example, based in part on each selected sensor meeting threshold of responsivity to the test gas. Additionally, the subset of gas sensors can be selected based in part on each selected sensor being below a threshold recovery period after termination of exposure of the selected gas sensor to the test gas.

A gas sensor can have no response or a response below a threshold response to a particular analyte. A gas sensor can have a threshold response to a particular analyte. In some implementations, a change in electrical resistivity of a gas sensor 108 is measured prior to exposure to the test gas, during exposure to the test gas, and after termination of exposure to the test gas. A plot of the response of the gas sensor versus time of collection can be recorded for each gas sensor by the data processing apparatus.

In some implementations the multiple gas sensors are 38 or more gas sensors in the multi-modal array of gas sensors 108, where the subset of gas sensors includes fewer than all of the total number of gas sensors in the gas sensing apparatus 102. For example, the total number of gas sensors in the gas sensing apparatus 102 is 30 gas sensors of multiple different types, e.g., MOx sensors, PID sensors, electrochemical sensors, and the subset of selected gas sensors are 15 gas sensors, 20 gas sensors, or 8 gas sensors.

The subset of gas sensors that is selected represents an optimized subset of the total available gas sensors in the gas sensing apparatus 102 for responding to the set of analytes associated with the object 105. For example, a full set of gas sensors can include gas sensors 108a-108h, as depicted in FIG. 1, while the selected subset can include 108a, 108c, 108d, and 108h.

In some implementations, the subset of gas sensors 108 can include sensors that are responsive to at least one of the multiple analytes associated with the object 105. Each of the gas sensors of the subset of gas sensors 108 can be responsive to one or more of the multiple analytes. For example, gas sensor 108a can be responsive to a first analyte, gas sensor 108c can be responsive to the first analyte and a second analyte, gas sensors 108d and 108h can both be responsive to the second analyte and a third analyte.

In some implementations, the subset of gas sensors 108 can include sensors that are unresponsive to one or more of the multiple analytes of the test gas. Sensors that are unresponsive may have zero response to a particular analyte or can have a response to an analyte below a threshold responsivity. One or more of the gas sensors of the subset of gas sensors 108 can be unresponsive to one or more of the multiple analytes associated with the object 105. Continuing the example from above, gas sensor 108a can be unresponsive to the second and third analyte, gas sensor 108c can be unresponsive to the third analyte, and gas sensors 108d and 108h are unresponsive to the first analyte.

In some implementations, the subset of gas sensors that each meet the threshold response can include meeting a threshold reactivity to one or more analytes of the multiple analytes in the test gas. In one example, the threshold reactivity includes a threshold change in resistivity of the gas sensor in response to the one or more analytes, e.g., for a MOx sensor. In another example, the threshold reactivity includes a threshold oxidation or reduction of the one or more analytes at an electrode of the sensor, e.g., for an electrochemical sensor. The threshold reactivity can be defined by a change of at least 0.1% relative to a total response range of a particular gas sensor. The threshold reactivity can be defined in part by what is considered a standard detectable signal for the particular gas sensor and can be different depending on the total response range of the particular gas sensor, e.g., can be different between a MOx sensor and an electrochemical sensor.

Selecting a proper subset further includes determining to include the selected gas sensor in the proper subset of gas sensors based on the selected gas sensor meeting a threshold temporal response, for example, an amount of time between exposure of a particular gas sensor to the test gas and the particular gas sensor reaching the threshold response,

The threshold temporal response can further include an amount of recovery time for the particular gas sensor to reach a baseline reading after termination of exposure to the test gas. In other words, an amount of time it takes for the gas sensor to recover from exposure to the test gas before it can be exposed again to another test gas.

The imaging data is annotated with an object annotation label by the data processing apparatus and using the set of sample data (512). The imaging data 121 can be annotated with the objects 105 appearing in the imaging 121 and further can be annotated with object annotation labels, for example, a classification of the object 105, a relative position of the object 105 from the gas inlet 110, or the like.

Training data is generated from the set of sample data and the labeled imaging data for the test gas representative of the object of interest within the first environment (514). In some implementations, training data is generated by recording the captured response for each sensor to the test gas, an amount of time that the response was captured, e.g., a “sniff” time, as well as an amount of time that a baseline measurement of no gas exposure was recorded prior to exposure of the test gas and an amount of time that the response was captured after termination of the exposure to the test gas. In other words, training data includes sensor response as well as labeled timestamps denoting baseline measurement, exposure to test gas, and recovery measurement. Each labeled sensor response is produced for various test gases including varying compositions of analytes and concentrations of each analyte.

The process described with reference to Steps 502-512 can be repeated for multiple test gases, where each test gas is representative of the test environment. The multiple test gases can be selected based on a range of compositions and/or environmental conditions, e.g., temperature, over which the response of the gas sensors 108 are measured to generate the training data.

In some implementations, prior to exposing the gas sensors to another test gas, the multiple gas sensors 108 are exposed to a purge gas, e.g., nitrogen or compressed clean dry air, for a period of time. The purge gas, e.g., gas source 130a depicted in FIG. 1, can be used to assist in shortening a recovery time of the sensors after exposure to the test gas, and/or to ensure that no remaining test gas is present within the housing 104 or gas manifold 126.

In some implementations, the period of time of exposure of the multiple gas sensors 108 to the purge gas can be an amount of time for each of the sensors to reach a baseline resistivity reading. The period of time of exposure can be selected based on a longest recovery time of all the respective recovery times for the multiple gas sensors 108. The period of time exposure of the multiple gas sensors 108 to the purge gas can be, for example, 1 minute, 5 minutes, 30 seconds, or the like.

Training data is provided to a machine-learned model (516). A machine-learned model can be trained for each intended test environment using a particular set of objects and respective associated test gases and environmental conditions. For example, a machine-learned model for an industrial applications can be different that a machine-learned model for a food production facility, where both sets of training data are generated using system 100 described with reference to FIG. 1.

FIG. 6 is a flow diagram of another example process 600 of the gas sensing apparatus. Imaging data is received from a camera (602). Referring to FIG. 2, a gas sensing apparatus 202 is located in a test environment, e.g., deployed in a factory-setting, in an agricultural setting, or the like. In one example, the gas sensing apparatus 202 is deployed in an apple orchard. The camera 117 collects imaging data 121 of a portion of the test environment within a field of view of the camera, e.g., including at least an area surrounding the gas inlet 110 of the gas sensing apparatus 202. Imaging data 121 can be collected by the camera 117 over a period of time, e.g., at periodic intervals for a given amount of time. Imaging data 121 can be collected before each “sniff” measurement by the gas sensing apparatus 202. In some implementations, camera 117 may continuously or semi-continuously collect imaging data 121 of the test environment.

Referring back to FIG. 6, an object of interest is identified in a test environment from the imaging data including one or more object annotation labels of the object of interest (604). As described with reference to FIG. 1 above, imaging data 121 can be processed by image processing apparatus 119 to identify one or more objects 105 in the test environment, wherein the image processing module 123 may use various different image processing software techniques to identify objects and classify the objects.

The image processing apparatus 119 can further identify one or more object annotation labels of the object of interest 105. Object annotation labels include a classification of the object and physical features of the object, e.g., a yellow banana, a red apple, etc.

A subset of gas sensors of multiple gas sensors and a set of performance parameters are selected based on the object of interest and the one or more object annotation labels (606). As described with reference to FIGS. 1 and 5, a machine-learned model is trained using labeled imaging data 125, response data 118, as well as composition data 122 and the like. The machine-learned model can be therefore trained to identify a subset of gas sensors of the multiple gas sensors 108 of the gas sensing apparatus 102 and a set of performance parameters based in part on the object of interest 105 and one or more object annotation labels of the object of interest 105 that are identified in the collected imaging data 121.

In some implementations, the subset of gas sensors selected by the machine-learned model is a proper subset of the multiple gas sensors 108 of the gas sensing apparatus, where the proper subset includes only gas sensors that are sensitize to a set of analytes associated with the object of interest 105 including one or more object annotation labels. For example, a proper subset of gas sensors may be sensitive to detect ethylene for an object that is identified as a banana with associated object characteristic of “ripe banana.”

The set of performance parameters can be selected based in part on a distance of the object of interest 105 from the gas inlet 110 of the gas sensing apparatus 102. For example, a sensitivity of the gas sensors might be increased for an object 105 that is determined to be located farther away from the gas inlet 110 relative to if the object 105 is located closer to the gas inlet 110. The distance of the object 105 from the gas inlet can be determined using the imaging data 121 captured by the camera 117 of the test environment including the object 105.

In some implementations, the set of performance parameters can be selected based in part on an air flow rate at the gas inlet 110. For example, a collection time for the gas sensors, e.g., an amount of time the gas sensors are exposed to the test gas, may be adjusted based on an air flow rate at the gas inlet 110. In one example, a collection time may be increased for a lower air flow rate as compared to a faster air flow rate.

In some implementations, the set of performance parameters can be selected based in part on a relative toxicity of the object of interest 105. A sensitivity of the gas sensors, e.g., a detection threshold, may be set based on a relative toxicity of one or more analytes associated with the object of interest 105. For example, an object with an associated known toxic analyte may result in the selection of a higher sensitivity of the gas sensors relative to an object without a known toxic analyte.

In some implementations, the set of performance parameters can be selected based on a relative sensitivity of the gas sensors 108 to one or more analytes of the object of interest 105. In one example, an operating temperature of a gas sensor may be adjusted in response to a sensitivity of the gas sensor to a particular analyte of interest associated with the object 105, e.g., operating the gas sensor at a higher temperature for an analyte for which the gas sensors is sensitive relative to a lower temperature for an analyte for which the gas sensor is more sensitive. In another example, a collection time for the gas sensors may be adjusted based on a sensitivity of the gas sensors to the particular analyte of the object of interest 105, e.g., increase the collection time for an analyte for which the gas sensor is less sensitive relative to an analyte for which the gas sensor is more sensitive.

The gas sensors are exposed to a test gas from the test environment including the object of interest (608). Referring to FIG. 2, the gas sensors 108 are exposed to the test gas 204 from the test environment including the object 105 via the gas inlet 110. As described above, the test gas 204 may first pass through an environmental regulator 106 to adjust one or more of a temperature and relative humidity of the test gas 204 before reaching the gas sensor array 108. The test gas 204 flows across the gas sensors 108 and is exhausted via a gas outlet 112. In some implementations, a fan 114 may be utilized to generate a negative pressure within the housing 104 to pull the test gas across the gas sensors 108 and evacuate the test gas from the housing 104 via the gas outlet 112.

Response data is collected from each of the gas sensors of the subset of gas sensors (610). Response data collected from the selected subset of gas sensors can then be provided by data processing apparatus 116 to the trained machine-learned model to identify information 208, e.g., characteristics descriptive of the object 105 and/or the test environment surrounding the gas sensing apparatus 202. Information about the object of interest 105 can include a composition of analytes detected in the test environment including identifying the analytes present and respective concentrations of the analytes that are present in the test environment and associated with the object of interest 105. For example, the data processing apparatus 116 can provide the response data, and identified object 105 and object annotation labels to the machine learned model and, based on the identified object 105, e.g., a banana, and the response data collected by the subset of sensors 108, that the banana is ripe.

In another example, an identified object 105 may be a plume of smoke emitting from a piece of equipment, where object annotation labels can include color, texture, dimensions, etc. of the plume of smoke. The response data collected from the subset of gas sensors can be provided to the machine-learned model along with the identified object and object annotation labels and information about the plume of smoke, e.g., toxic vs. non-toxic, one or more analytes in the plume of smoke, etc., can be identified.

In some implementations, the information 208 determined from the response data and imaging data can be provided for display to the user, e.g., using display 206. As described with reference to FIG. 2, information 208 presented in the display 206 can include test results including compositional data for the test environment including the object 105. The information 208 can further include one or more alerts, e.g., hazard warnings, or other regulatory alerts to the user.

In some implementations, one or more objects are identified in the imaging data 121 that are not of interest in the test environment in addition to an object of interest 105 in the test environment. For example, an object of interest is particular plant species and an object not of interest is a weed. A modified subset of gas sensors from the array of gas sensors 108 of the gas sensing apparatus 202 can be selected based on the identified objects not of interest and the object annotation labels of the objects not of interest as well as the objects of interest and object annotation labels of the objects of interest. Continuing the example, a modified subset of gas sensors may be selected to detect only known analytes associated with the objects of interest and not known analytes associated with the objects not of interest or not known analytes that are in common between the two. In other words, the modified subset of gas sensors may be selected to look for differentiator gases between the objects of interest and objects not of interest.

In some implementations, the modified set of gas sensors are sensitive to one or more known analytes associated with the object of interest 105 and are not sensitive to one or more known analytes of the object not of interest. The modified set of gas sensors can be selected based in part on the one or more object annotation labels for the objects of interest and objects not of interest. For example, a gas sensor may be selected that is only sensitive to a known analyte (e.g., a VOC) associated with wine grapes and not sensitive to a known analyte associated with a pesticide.

In some implementations, a modified set of performance parameters can be selected based on the identified objects not of interest in the test environment captured by the imaging data 121. The modified performance parameters may be selected to differentiate between the known analytes associated with the objects of interest and the known analytes associated with the objects not of interest.

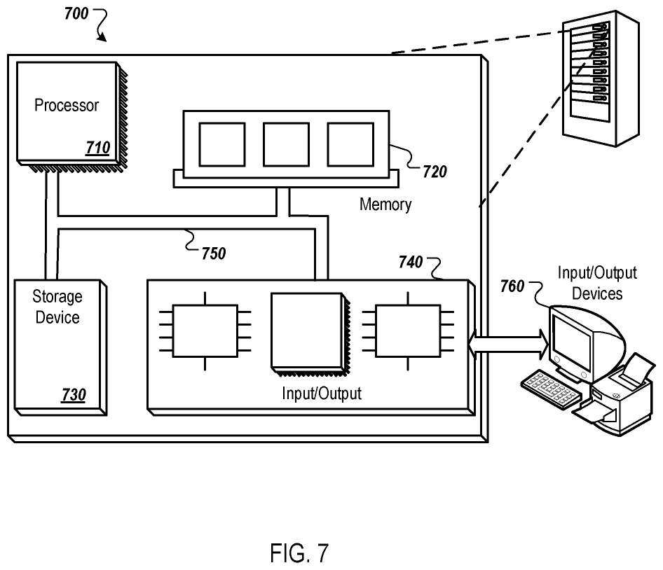

FIG. 7 is a block diagram of an example computer system 700 that can be used to perform operations described above. The system 700 includes a processor 710, a memory 720, a storage device 730, and an input/output device 740. Each of the components 710, 720, 730, and 740 can be interconnected, for example, using a system bus 750. The processor 710 is capable of processing instructions for execution within the system 700. In one implementation, the processor 710 is a single-threaded processor. In another implementation, the processor 710 is a multi-threaded processor. The processor 710 is capable of processing instructions stored in the memory 720 or on the storage device 730.

The memory 720 stores information within the system 700. In one implementation, the memory 720 is a computer-readable medium. In one implementation, the memory 720 is a volatile memory unit. In another implementation, the memory 720 is a non-volatile memory unit.

The storage device 730 is capable of providing mass storage for the system 700. In one implementation, the storage device 730 is a computer-readable medium. In various different implementations, the storage device 730 can include, for example, a hard disk device, an optical disk device, a storage device that is shared over a network by multiple computing devices (for example, a cloud storage device), or some other large capacity storage device.

The input/output device 740 provides input/output operations for the system 700. In one implementation, the input/output device 740 can include one or more network interface devices, for example, an Ethernet card, a serial communication device, for example, a RS-232 port, and/or a wireless interface device, for example, a 802.11 card. In another implementation, the input/output device can include driver devices configured to receive input data and send output data to other input/output devices, for example, keyboard, printer and display devices 760. Other implementations, however, can also be used, such as mobile computing devices, mobile communication devices, set-top box television client devices, etc.

Although an example processing system has been described in FIG. 7, implementations of the subject matter and the functional operations described in this specification can be implemented in other types of digital electronic circuitry, or in computer software, firmware, or hardware, including the structures disclosed in this specification and their structural equivalents, or in combinations of one or more of them.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, that is, one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, for example, a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, for example, an FPGA (field programmable gate array) or an ASIC (application-specific integrated circuit). The apparatus can optionally include, in addition to hardware, code that creates an execution environment for computer programs, for example, code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, for example, an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a Global Positioning System (GPS) receiver, or a portable storage device, for example, a universal serial bus (USB) flash drive, to name just a few.

Computer-readable media suitable for storing computer program instructions and data include all forms of non-volatile memory, media and memory devices, including by way of example semiconductor memory devices, for example, EPROM, EEPROM, and flash memory devices; magnetic disks, for example, internal hard disks or removable disks; magneto-optical disks; and CD-ROM and DVD-ROM disks.

To provide for interaction with a user, embodiments of the subject matter described in this specification can be implemented on a computer having a display device, for example, a CRT (cathode ray tube) or LCD (liquid crystal display) monitor, for displaying information to the user and a keyboard and a pointing device, for example, a mouse or a trackball, by which the user can provide input to the computer. Other kinds of devices can be used to provide for interaction with a user as well; for example, feedback provided to the user can be any form of sensory feedback, for example, visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of messages to a personal device, for example, a smartphone that is running a messaging application and receiving responsive messages from the user in return.

Data processing apparatus for implementing machine learning models can also include, for example, special-purpose hardware accelerator units for processing common and compute-intensive parts of machine learning training or production, that is, inference, workloads.

Machine learning models can be implemented and deployed using a machine learning framework, for example, a TensorFlow framework, a Microsoft Cognitive Toolkit framework, an Apache Singa framework, or an Apache MXNet framework.

Embodiments of the subject matter described in this specification can be implemented in a computing system that includes a back-end component, for example, as a data server, or that includes a middleware component, for example, an application server, or that includes a front-end component, for example, a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, for example, a communication network. Examples of communication networks include a local area network (LAN) and a wide area network (WAN), for example, the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, for example, an HTML page, to a user device, for example, for purposes of displaying data to and receiving user input from a user interacting with the device, which acts as a client. Data generated at the user device, for example, a result of the user interaction, can be received at the server from the device.

While this specification contains many specific implementation details, these should not be construed as limitations on the scope of any features or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A multi-modal gas sensing apparatus comprising:
a camera configured to capture imaging data including at least a portion of a test environment, the test environment comprising the gas sensing apparatus and an object of interest within the field of view of the camera;
a plurality of gas sensors including a first type of gas sensor and a second type of gas sensor different from the first type of gas sensor, wherein each of the first type of gas sensor and second type of gas sensor is sensitive to a respective set of analytes;
a housing configured to hold the plurality of gas sensors;
a gas inlet coupled to the housing and configured to expose the plurality of gas sensors to a gas introduced from the test environment via the gas inlet; and
a data processing apparatus in data communication with the plurality of gas sensors and the camera, wherein the data processing apparatus is configured to perform the operations comprising:
receiving, from the camera, imaging data;
identifying, from the imaging data, the object of interest in the test environment and one or more object annotation labels;
selecting, based on the object of interest and one or more object annotation labels, a proper subset of the plurality of gas sensors and a set of performance parameters; and
collecting, for each gas sensor of the proper subset of gas sensors, response data from the exposure to the test gas.

2. The apparatus of claim 1, wherein selecting the proper subset of the plurality of gas sensors and the set of performance parameters comprises selecting only the gas sensors of the plurality of gas sensors that are sensitive to a plurality of analytes associated with the object of interest.

3. The apparatus of claim 1, wherein the set of performance parameters comprises an operating temperature of one or more of the proper subset of the plurality of gas sensors.

4. The apparatus of claim 3, wherein the set of performance parameters comprises a sensitivity level of one or more of the proper subset of gas sensors.

5. The apparatus of claim 1, wherein selecting the set of performance parameters is based in part on one or more of a distance of the object of interest from the gas inlet, an air flow rate at the gas inlet, a relative toxicity of the object of interest, and a relative sensitivity of the plurality of gas sensors to the object of interest.

6. The apparatus of claim 5, wherein the distance of the object of interest from the gas inlet is determined based on the imaging data including the object of interest.

7. The apparatus of claim 1, further comprising:
identifying, from the imaging data, one or more objects of not of interest in the test environment and one or more object annotation labels for the objects not of interest; and
selecting, based on the one or more objects of not of interest and one or more object annotation labels for the objects not of interest, a modified proper subset of the plurality of gas sensors and a modified set of performance parameters.

8. The apparatus of claim 1, further comprising:
identifying, based on the response data, one or more properties of the object of interest.

9. The apparatus of claim 1, further comprising a user interface including a touch-screen interface for a user to interact with the multi-modal gas sensing apparatus.

10. The apparatus of claim 9, wherein user interaction comprises identifying, by the user and by an indication on the touch-screen interface, one or more objects of interest in the field of view of the camera.

11. A method for training a multi-modal gas sensor array comprising:
generating training data for a plurality of test gases, each test gas comprising a plurality of analytes and introduced into a first environment by an object of interest located within the first environment, wherein for each test gas the generating of training data comprises:
collecting, by a camera configured to capture the object of interest within a field of view of the camera, imaging data including the object of interest located within the first environment;
exposing the multi-modal gas sensor array comprising a plurality of gas sensors to the test gas, wherein the plurality of gas sensors comprises a first type of gas sensor and a second type of gas sensor different from the first type of gas sensor;
collecting, by a data processing apparatus and from each of the plurality of gas sensors, a set of sample data comprising response data for each of the plurality of gas sensors responsive to the exposure of the test gas;
selecting, from the set of sample data, a subset of gas sensors from the plurality of gas sensors for the test gas, wherein the response data collected for each gas sensor of the subset of gas sensors meets a threshold response;
annotating, by the data processing apparatus and using the set of sample data, the imaging data with an object annotation label;
generating, from the set of sample data and the labeled imaging data, training data for the test gas representative of the object of interest within the first environment; and
providing, to a machine-learned model, the training data.

12. The method of claim 11, wherein the object annotation label comprises one or more of a distance of the object of interest from a gas inlet of the multi-modal gas sensor array, an air flow rate at the gas inlet, a relative toxicity of the object of interest, and a relative sensitivity of the plurality of gas sensors to the object of interest.

13. The method of claim 11, further comprising:
collecting, by the camera, imaging data including a particular object of interest within the field of view of the camera located within a test environment;
determining, by the data processing apparatus and from the imaging data, one or more object annotation labels for the particular object of interest;
identifying, by the data processing apparatus and using the machine-learned model, a subset of gas sensors from the plurality of gas sensors sensitive to one or more analytes associated with the particular object of interest based on the one or more object annotation labels;
exposing the multi-modal gas sensor array comprising the plurality of gas sensors to a test gas from the test environment including the particular object of interest;
collecting, by the data processing apparatus and from the subset of gas sensors, response data from each of the subset of gas sensors identified as sensitive to the one or more analytes associated with the particular object of interest; and
determining, by the data processing apparatus and using the machine-learned model, one or more characteristics descriptive of the particular object of interest within the test environment.

14. The method of claim 13, wherein the one or more characteristics descriptive of the particular object of interest comprises identifying respective concentrations of the one or more analytes associated with the particular object of interest.

15. The method of claim 13, wherein determining the one or more object annotation labels for the particular object of interest comprises determining a distance of the particular object of interest from the gas inlet of the multi-modal gas sensor array.

16. The method of claim 13, wherein determining one or more object annotation labels for the object of interest comprises performing image recognition analysis on the imaging data collected by the camera.

17. The method of claim 13, further comprising:
receiving, from a user, a user interaction via a touch-screen interface of the multi-modal gas sensor array, wherein the user interaction comprises identifying, by the user and by an indication on the touch-screen interface, one or more particular objects of interest in the field of view of the camera.

18. The method of claim 13, further comprising:
determining, by the data processing apparatus and from the imaging data, one or more objects not of interest within the field of view of the camera;
determining, by the data processing apparatus and from the imaging data, one or more object annotation labels for the one or more objects of not of interest; and
identifying, by the data processing apparatus and using the machine-learned model, a modified subset of gas sensors from the plurality of gas sensors,
wherein the modified subset of gas sensors are sensitive to one or more analytes associated with the particular object of interest based on the one or more object annotation labels for the object of interest,
and are not sensitive to one or more analytes associated with the one or more objects not of interest based on the one or more object annotations labels for the one or more objects not of interest.

19. The method of claim 13, wherein identifying the subset of gas sensors from the plurality of gas sensors sensitive to one or more analytes associated with the particular object of interest based on the one or more object annotation labels further comprises:
selecting, by the data processing apparatus, performance parameters for the subset of gas sensors comprising an operating temperature of one or more of the gas sensors of the subset gas sensors.

20. The method of claim 19, wherein the set of performance parameters comprises a sensitivity level of one or more of the gas sensors of the subset of gas sensors.
